(19) United States
(12) Patent Application Publication
(10) Pub. No.: US 2008/0081776 A1
(43) Pub. Date: Apr. 3, 2008

(54) MILD FOAMING PERSONAL CLEANSING COMPOSITION WITH HIGH LEVELS OF HYDROCARBON WAX AND OIL EMOLLIENTS

(75) Inventors: Brian Andrew CROTTY, Branford, CT (US); Alexander Kingston SHUTAK, Guilford, CT (US); Thomas Nikolaos MORIKIS, Southington, CT (US); Virgilio Barba VILLA, Emerson, NJ (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: CONOPCO, INC., D/B/A UNILEVER, Englewood Cliffs, NJ (US)

(21) Appl. No.: 11/536,383

(22) Filed: Sep. 28, 2006

(51) Int. Cl. A61K 8/00

(52) U.S. Cl. 510/130

(57) ABSTRACT

A liquid cleansing composition is described that contains high levels of hydrocarbon wax and oil emollients yet produces substantial levels of foaming. The cleansing composition contains C6 to 20 acyl sarcosinate surfactant(s) and total hydrocarbon wax and oil emollients in a specific ratio range and preferably has a liquid crystalline structure. The inventive composition shows excellent stability.

### BACKGROUND OF THE INVENTION

1. Field of the Invention

The present invention relates to detergent compositions suitable for topical application for cleansing and moisturizing the human body, such as the skin and hair. In particular, it relates to very mild, high foaming personal cleansing compositions with substantial levels of hydrocarbon wax or oil emollients or blends thereof.

2. Background of the Art

Prior art skin cleansers modify the way the skin feels after the shower by depositing materials such as oils or polymers. However, such cleansers often have disadvantageous sensory or physical properties such as a slimy feel and/or poor lather. Stability problems are also frequently observed with many prior art cleansers containing combinations of substantial amounts of hydrophobic emollients for skin conditioning, such as hydrocarbon wax and oil emollients and surfactants in sufficient quantity to produce good foaming.

U.S. Pat. No. 6,903,057 issued on Jun. 7, 2005 to Tsaur describes a liquid cleansing composition containing high levels of hydrophobic emollients with relatively low levels of surfactants and stabilized with a starch structuring system.

U.S. Pat. No. 6,906,016 issued on Jun. 14, 2005 to Tsaur describes a liquid cleansing composition containing high levels of hydrophobic emollients with relatively low levels of surfactants and stabilized with a combined fatty acid and starch structuring system.

Surprisingly it has been discovered that by incorporating a specific amount of N—C6 to C₂₀ acyl sarcosinate(s) and a specific amount of total hydrocarbon wax or oil emollients or blends thereof in a selected ratio range, a cleansing composition of excellent foam ability and stability results that was exceedingly mild and moisturizing.

### BRIEF DESCRIPTION OF THE INVENTION

In one aspect of the invention is a stable cleansing composition including but not limited to:

a. about 0.5 to 20 % by wt. of total N—(C6-C20) acyl sarcosinate surfactant(s);

b. about 0.5 to 50 % by wt. of total hydrocarbon wax or oil emollients or blends thereof;

c. greater than about 0.5 % by wt. of C10 to C18 fatty acid(s);

d. about 5 to 95 % by wt. of water; and

e. wherein the ratio of the sarcosinate surfactant to total hydrocarbon wax and oil emollients is in the range of about 0.04 to 2.0.

### DETAILED DESCRIPTION OF THE INVENTION

All publications and patent applications patents, and other references mentioned herein are incorporated by reference in their entirety.

In one aspect of the invention is a stable cleansing composition including but not limited to:

a. about 0.5 to 20 % by wt. of total N—(C6-C20) acyl sarcosinate surfactant(s);

b. about 0.5 to 50 % by wt. of total hydrocarbon wax or oil emollients or blends thereof;

c. greater than about 0.5 % by wt. of C10 to C18 fatty acid(s);

d. about 5 to 95 % by wt. of water; and

e. wherein the ratio of the sarcosinate surfactant to total hydrocarbon wax and oil emollients is in the range of about 0.04 to 2.0.

Advantageously the inventive cleansing composition has a liquid crystal structured phase such as a lamellar, cubic or hexagonal structured liquid crystal structured phase. Preferably it is lamellar. Most preferably its viscosity value is in the range of about 8 KPaS to 800 KPaS at 25 C using the Standard Viscosity Method. Advantageously the composition remains stable under at least one of the Standardized Stability tests described below.

Preferably the composition further includes greater than about 0.5, 1, 2, 3, or 5 % by wt. of hydrophobic emollient(s) selected from glyceride oil(s), polybutenes with a number average degree of polymerization of about 3 to about 110, silicone oils and blends thereof. In a preferred embodiment the total hydrocarbon wax and oil emollients blend has an observed melting point in the range of about 40 to 70 C, more preferably with a minimum melting point of 45 or 50 C and a maximum melting point of 55 or 60 C and in a further preferred embodiment a melting point range of about 51.6 to 57.2 C.

Advantageously the inventive cleansing composition provides a foam volume of greater than or equal to 20, 22, 25, 27, or 30mis using the standard foam determination method described below. Preferably the inventive composition further includes about 3 to 30 % by wt. of total anionic, amphoteric and cationic surfactant(s) or blends thereof not including the N—(C6-C20) acyl sarcosinate surfactant(s). More preferably with a total minimum level of 0.5, 1 or 1.3% and a total maximum level of 25, 40 or 60% by wt. of total anionic, amphoteric and cationic surfactant(s). In a further preferred embodiment the inventive cleansing composition further contains about 0.05 to 10% by wt. of cationic polymer(s).

Hydrocarbon wax and oil emollients as the term is used in the invention are defined as not including polybutenes with a number average degree of polymerization of about 3 to about 110 such as e.g. Indopol H300/1500 polyisobutenes. The polybutenes excluded from the definition of hydrocarbon wax and oil emollients are synthetic hydrocarbon polymers made via acid catlayzed cationic polymerization of an isobutene-rich C4 stream and are essentially pure polyisobutene but also have some n-butene incorporated. Each molecule also possesses an olefinic double bond at or near one end and has the following generic structure:

H₃CC(CH₃)₂[CH₂C(CH₃)₂]ₙ[CH₂]ₘC(R¹)(R²)C(R³)═C(R⁴)(R⁵)

Where
m=0 or 1
n=1-ca. 110
R¹-R⁴ is H or CH3
R⁵ is H, CH₃, C₂H₅, OR C₃H₇.

Although not included in the definition of hydrocarbon wax and oil emollients, such polybutene compounds may be advantageously incorporated in the inventive composition, preferably at minimum levels of about 0.5, 11 2, 3, or 5 % bywt.

Surfactants:

Surfactants are an essential component of the inventive cleansing composition. They are compounds that have hydrophobic and hydrophilic portions that act to reduce the surface tension of the aqueous solutions they are dissolved in. In addition to the surfactants required by the invention, other useful surfactants can be added to the inventive composition and can include anionic, non-ionic, amphoteric, and cationic surfactants, and blends thereof.

Anionic Surfactants:

The cleansing composition of the present invention contains N—(C₆-C₂₀) acyl Sarcosinate surfactants and optionally other anionic surfactants. Preferably the contains N—(C₆-C₂₀) acyl Sarcosinate surfactants are preferably used in the range of about 0.3% to 20% by wt. and more preferably in the range of about 0.5% to 12%by wt.

Sarcosinates required for the invention are generally indicated by the formula:

R¹CON(CH₃)CH₂CO₂M,

wherein R¹ ranges is C₆-C₂₀ acyl and M is a solubilizing cation. Anionic surfactants other than N—(C6-C₂₀) acyl Sarcosinate surfactants may be used. They are advantageously employed at a total minimum level of about 0.5, 1, or 1.3% by wt. and a total maximum level of about 25, 40, or 60% by wt. Examples of useful anionic surfactants include the following.

Monoalkyl sulfosuccinates having the formula:

R⁴O₂CCH₂CH(SO₃M)CO₂M

may be usefully employed in the invention as described above wherein R⁴ ranges from C₁₀-C₁₆ alkyl and M is a solubilizing cation.

Other anionic detergent actives which may be used include aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic may also be an alkyl sulfate (e.g., C₁₂-C₁₈ alkyl sulfate) or alkyl ether sulfate (including alkyl glyceryl ether sulfates). Among the alkyl ether sulfates are those having the formula:

RO(CH₂CH₂O)ₙSO₃M

wherein R is an alkyl or alkenyl having 8 to 18 carbons, preferably 12 to 18 carbons, n has an average value of greater than 1.0, preferably greater than 3; and M is a solubilizing cation such as sodium, potassium, ammonium or substituted ammonium. Ammonium and sodium lauryl ether sulfates are preferred.

The anionic may also include dialkyl sulfosuccinates (e.g., C₆-C₂₂ sulfosuccinates); alkyl and acyl taurates, sulfoacetates, C₈-C₂₄ monoalkyl or dialkyl phosphates, n-acyl amino acid surfactant(s) alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Amide-MEA sulfosuccinates of the formula;

R⁴CONHCH₂CH₂O₂CCH₂CH(SO₃M)CO₂M

May be used wherein R⁴ ranges from C₈-C₂₂ alkyl and M is a solubilizing cation may be used.

Taurates are generally identified by formula:

R²CONR³CH₂CH₂SO₃M

wherein R² ranges from C₈-C₂₀ alkyl, R³ ranges from C₁-C₄ alkyl and M is a solubilizing cation.

The inventive cleansing composition may contain C₈-C₁₈ acyl isethionates. These esters are prepared by reaction between alkali metal isethionate with mixed aliphatic fatty acids having from 6 to 18 carbon atoms and an iodine value of less than 20. At least 75% of the mixed fatty acids have from 12 to 18 carbon atoms and up to 25% have from 6 to 10 carbon atoms.

The acyl isethionate may be an alkoxylated isethionate such as is described in Ilardi et al., U.S. Pat. No. 5,393,466, titled “Fatty Acid Esters of Polyalkoxylated isethonic acid; issued Feb. 28, 1995; hereby incorporated by reference. This compound has the general formula:

R C—O(O)—C(X)H—C(Y)H₂—(OCH—CH₂)ₘ—SO₃M⁺

wherein R is an alkyl group having 8 to 18 carbons, m is an integer from 1 to 4, X and Y are hydrogen or an alkyl group having 1 to 4 carbons and M⁺ is a monovalent cation such as, for example, sodium, potassium or ammonium.

Amphoteric Surfactants

One or more amphoteric surfactants may be used in this invention. Amphoteric surfactants are preferably used at levels as low as 0.5, 1, 2, 3, 4 or 5 % by wt. and at levels as high as 6, 8, 10, 12, 15, 25, 40 or 60% by wt.

Such surfactants include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula:

R¹—[—C(O)—NH(CH₂)ₙ—]ₘ—N⁺—(R²)(R³)X—Y

where R¹ is alkyl or alkenyl of 7 to 18 carbon atoms;
R² and R³ are each independently alkyl, hydroxyalkyl or carboxyalkyl of 1 to 3 carbon atoms;
n is 2 to 4;
m is 0 to 1;
X is alkylene of 1 to 3 carbon atoms optionally substituted with hydroxyl, and
Y is —CO₂— or —SO₃—

Suitable amphoteric surfactants within the above general formula include simple betaines of formula:

R¹—N⁺—(R²)(R³)CH₂CO₂⁻

and amido betaines of formula:

R¹—CONH(CH₂)ₙ—N⁺—(R²)(R³)CH₂CO₂⁻
where n are 2 or 3.

In both formulae R¹, ² and R³ are as defined previously. R¹ may in particular be a mixture of C₁₂ and C₁₄ alkyl groups derived from coconut oil so that at least half, preferably at least three quarters of the groups R¹ have 10 to 14 carbon atoms. R² and R³ are preferably methyl.

A further possibility is that the amphoteric detergent is a sulphobetaine of formula:

R¹—N⁺—(R²)(R³)(CH₂)₃SO₃⁻

or

R¹—CONH(CH₂)ₘ—N⁺—(R²)(R³)(CH₂)₃SO₃⁻

where m is 2 or 3, or variants of these in which —(CH₂)₃SO₃ is replaced by

—CH₂C(OH)(H)CH₂SO₃⁻

In these formulae R¹, R² and R³ are as discussed previously.

Amphoacetates and diamphoacetates are also intended to be covered in possible zwitterionic and/or amphoteric compounds which may be used such as e.g., sodium lauroamphoacetate, sodium cocoamphoacetate, and blends thereof, and the like.

Nonionic Surfactants

One or more nonionic surfactants may also be used in the cleansing composition of the present invention. Nonionic surfactants are preferably used at levels as low as 0.5, 1, 2, 3 or 5% by wt. and at levels as high as 6, 8, 10, 12 or 15% by wt.

The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic detergent compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxide, and the like.

The nonionic may also be a sugar amide, such as a polysaccharide amide. Specifically, the surfactant may be one of the lactobionamides described in U.S. Pat. No. 5,389,279 to Au et al. titled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Cationic Skin Conditioning Agents

A useful component in compositions according to the invention is a cationic skin feel agent or polymer, such as for example cationic celluloses. Cationic polymers are preferably used at levels as low as about 0.01, 0.05, 0.1, 0.5, 1 or 2% and at levels as high as about 2, 3, 4 or 5% by wt.

Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the trade name Polymer LM-200.

A particularly suitable type of cationic polysaccharide polymer that can be used is a cationic guar gum derivative, such as guar hydroxypropyltrimonium chloride (Commercially available from Rhone-Poulenc in their JAGUAR trademark series). Examples are JAGUAR C13S, which has a low degree of substitution of the cationic groups and high viscosity, JAGUAR C15, having a moderate degree of substitution and a low viscosity, JAGUAR C17 (high degree of substitution, high viscosity), JAGUAR C16, which is a hydroxypropylated cationic guar derivative containing a low level of substitute groups as well as cationic quaternary ammonium groups, and JAGUAR 162 which is a high transparency, medium viscosity guar having a low degree of substitution.

Particularly preferred cationic polymers are JAGUAR C135, JAGUAR C15, JAGUAR C17 and JAGUAR C16 and JAGUAR C162, especially Jaguar C13S. Other cationic skin feel agents known in the art may be used provided that they are compatible with the inventive formulation.

Cationic Surfactants

One or more cationic surfactants may also be used in the cleansing composition. Cationic surfactants may be used at levels as low as about 0.01, 0.05, 0.1, 0.5, and 1% by wt. and at levels as high as 2, 3, 4 or 5% by wt. or as high as 6, 8, 10, 12, 15, 25, 40 or 60% by wt.

Examples of cationic detergents are the quaternary ammonium compounds such as alkyldimethylammonium halogenides. Other suitable surfactants which may be used are described in U.S. Pat. No. 3,723,325 to Parran Jr. titled “Detergent Compositions Containing Particle Deposition Enhancing Agents” issued Mar. 27, 1973; and “Surface Active Agents and Detergents” (Vol. I & II) by Schwartz, Perry & Berch, both of which are also incorporated into the subject application by reference.

In addition, the inventive cleansing composition of the invention may include 0 to 15% by wt. optional ingredients as follows. perfumes; sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer) and the like; all of which are useful in enhancing the appearance or cosmetic properties of the product.

The compositions may further comprise antimicrobials such as 2-hydroxy-4,2′,4′trichlorodiphenylether (DP300); preservatives such as dimethyloldimethylhydantoin (Glydant XL1000), parabens, sorbic acid etc., and the like.

The compositions may also comprise coconut acyl mono- or diethanol amides as suds boosters and strongly ionizing salts such as sodium chloride and sodium sulfate may also be used to advantage.

Antioxidants such as, for example, butylated hydroxytoluene (BHT) and the like may be used advantageously in amounts of about 0.01% or higher if appropriate.

Moisturizers (also known as hydrophilic emollients) that also are Humectants such as polyhydric alcohols, e.g. glycerin and propylene glycol, and the like; and polyols such as polyethylene glycols may be used.

Hydrocarbon wax and oil emollients are hydrophobic emollients that are used in the invention. Other hydrophobic emollients may be optionally used at levels that do not alter the unique sensory properties of the invention.

The term “emollient” (also considered skin conditioning compounds according to the invention) is defined as a substance which softens or improves the elasticity, appearance, and youthfulness of the skin (stratum corneum) by either increasing its water content, adding, or replacing lipids and other skin nutrients; or both, and keeps it soft by retarding the decrease of its water content.

Useful hydrophobic emollients include the following:

(a) silicone oils and modifications thereof such as linear and cyclic polydimethylsiloxanes; amino, alkyl, alkylaryl, and aryl silicone oils;

(b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride;

(c) natural waxes such as carnauba, spermaceti, beeswax, lanolin, and derivatives thereof;

(d) hydrophobic and hydrophilic plant extracts;

(e) inventive hydrocarbon wax and oil emollients include branched and unbranched hydrocarbons such as petrolatum, mineral oil, microcrystalline waxes, paraffins, ceresin, ozokerite, polyethylene, perhydrosqualene, paraffin oil, pristane, squalane, squalene, and combinations thereof and the like. Preferably the hydrocarbon wax and oil emollients include petrolatum and/or blends of microcrystalline wax and mineral oil and are advantageously present at levels of 10, 20, 30, 40, 50, 60, 70, 80, 90, 95% by wt. or more of the total hydrocarbon wax and oil emollients used. Most preferably petrolatum or another hydrocarbon oil/wax blend which has substantially equivalent skin protective properties to petrolatum as measured by art recognized and equivalent techniques is used alone.

(f) higher fatty acids such as lauric, myristic, palmitic, stearic, behenic, oleic, linoleic, linolenic, lanolic, isostearic, arachidonic and poly unsaturated fatty acids (PUFA);

(g) higher alcohols such as lauryl, cetyl, stearyl, oleyl, behenyl, cholesterol and 2-hexydecanol alcohol;

(h) fatty esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate;

(i) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils;

(j) mixtures of any of the foregoing components, and the like.

Ordered Liquid Crystalline Compositions:

The inventive cleansing composition preferably possesses ordered liquid crystalline microstructure, more preferably cubic, hexagonal or lamellar microstructure and most preferably lamellar microstructure. The rheological behavior of all surfactant solutions, including liquid cleansing solutions, is strongly dependent on the microstructure, i.e., the shape and concentration of micelles or other self-assembled structures in solution.

When there is sufficient surfactant to form micelles (concentrations above the critical micelle concentration or CMC), for example, spherical, cylindrical (rod-like or discoidal), spherocylindrical or ellipsoidal micelles may form. As surfactant concentration increases, ordered liquid crystalline phases such as lamellar phase, hexagonal phase, cubic phase or L3 sponge phase may form. The lamellar phase, for example, consists of alternating surfactant bilayers and water layers. These layers are not generally flat but fold to form submicron spherical onion like structures called vesicles or liposomes. The hexagonal phase, on the other hand, consists of long cylindrical micelles arranged in a hexagonal lattice. In general, the microstructure of most personal care products consist of either spherical micelles; rod micelles; or a lamellar dispersion.

As noted above, micelles may be spherical or rod-like. Formulations having spherical micelles tend to have a low viscosity and exhibit Newtonian shear behavior (i.e., viscosity stays constant as a function of shear rate; thus, if easy pouring of product is desired, the solution is less viscous and, as a consequence, it doesn't suspend as well). In these systems, the viscosity increases linearly with surfactant concentration.

Rod micellar solutions are more viscous because movement of the longer micelles is restricted. At a critical shear rate, the micelles align and the solution becomes shear thinning. Addition of salts increases the size of the rod micelles thereof increasing zero shear viscosity (i.e., viscosity when sitting in bottle) which helps suspend particles but also increases critical shear rate (point at which product becomes shear thinning; higher critical shear rates means product is more difficult to pour).

Lamellar dispersions differ from both spherical and rod-like micelles because they can have high zero shear viscosity (because of the close packed arrangement of constituent lamellar droplets), yet these solutions are very shear thinning (readily dispense on pouring). That is, the solutions can become thinner than rod micellar solutions at moderate shear rates.

In formulating liquid cleansing compositions, therefore, there is the choice of using rod-micellar solutions (whose zero shear viscosity, e.g., suspending ability, is not very good and/or are not very shear thinning); or lamellar dispersions (with higher zero shear viscosity, e.g. better suspending, and yet are very shear thinning). Such lamellar compositions are characterized by high zero shear viscosity (good for suspending and/or structuring) while simultaneously being very shear thinning such that they readily dispense in pouring. Such compositions possess a “heaping”, lotion-like appearance which conveys signals of enhanced moisturization.

When rod-micellar solutions are used, they also often require the use of external structurants to enhance viscosity and to suspend particles (again, because they have lower zero shear viscosity than lamellar phase solutions). For this, carbomers and clays are often used. At higher shear rates (as in product dispensing, application of product to body, or rubbing with hands), since the rod-micellar solutions are less shear thinning, the viscosity of the solution stays high and the product can be stringy and thick. Lamellar dispersion based products, having higher zero shear viscosity, can more readily suspend emollients and is typically creamier. In general, lamellar phase compositions are easy to identify by their characteristic focal conic shape and oily streak texture while hexagonal phase exhibits angular fan-like texture. In contrast, micellar phases are optically isotropic.

It should be understood that lamellar phases may be formed in a wide variety of surfactant systems using a wide variety of lamellar phase “inducers” as described, for example, in U.S. Pat. No. 5,952,286 issued to Puvvada, et al., on Sep. 14, 1999. Generally, the transitions from micelle to lamellar phase are functions of effective average area of head group of the surfactant, the length of the extended tail, and the volume of tail. Using branched surfactants or surfactants with smaller head groups or bulky tails are also effective ways of inducing transitions from rod micellar to lamellar.

One way of characterizing ordered liquid crystalline dispersions include measuring viscosity at low shear rate (using for example a Stress Rheometer) when additional inducer (e.g., oleic acid or isostearic acid) is used. At higher amounts of inducer, the low shear viscosity will significantly increase.

Another way of measuring ordered liquid crystalline dispersions is using freeze fracture electron microscopy. Micrographs generally will show ordered liquid crystalline microstructure and close packed organization of the lamellar droplets (generally in size range of about 2 microns).

In a preferred embodiment, the inventive ordered liquid crystalline phase composition preferably has a low shear viscosity in the range of about 2 to about 70 (mPa.S) More preferably the viscosity range is about 3 to about 50 (mPaS)

Optional Active Agents

Advantageously, active agents other than conditioning agents such as emollients or moisturizers defined above may be added to the cleansing composition in a safe and effective amount during formulation to treat the skin during the use of the product. These active ingredients may be advantageously selected from antimicrobial and antifungal actives, vitamins, anti-acne actives; anti-wrinkle, anti-skin atrophy and skin repair actives; skin barrier repair actives; non-steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; hair growth inhibitors; 5-alpha reductase inhibitors; desquamating enzyme enhancers; anti-glycation agents; topical anesthetics, or mixtures thereof; and the like.

These active agents may be selected from water soluble active agents, oil soluble active agents, pharmaceutically-acceptable salts and mixtures thereof Advantageously the agents will be soluble or dispersible in the cleansing composition. The term “active agent” as used herein, means personal care actives which can be used to deliver a benefit to the skin and/or hair and which generally are not used to confer a conditioning benefit, as is conferred by humectants and emollients previously described herein. The term “safe and effective amount” as used herein, means an amount of active agent high enough to modify the condition to be treated or to deliver the desired skin care benefit, but low enough to avoid serious side effects. The term “benefit,” as used herein, means the therapeutic, prophylactic, and/or chronic benefits associated with treating a particular condition with one or more of the active agents described herein. What is a safe and effective amount of the active agent ingredient will vary with the specific active agent, the ability of the active to penetrate through the skin, the age, health condition, and skin condition of the user, and other like factors. Preferably the composition of the present invention comprise from about 0.01% to about 50%, more preferably from about 0.05% to about 25%, even more preferably 0.1% to about 10%, and most preferably 0.1% % to about 5 by weight of the active agent component.

Anti-acne actives can be effective in treating acne vulgaris, a chronic disorder of the pilosebaceous follicles. Nonlimiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, mixtures thereof and the like.

Antimicrobial and antifungal actives can be effective to prevent the proliferation and growth of bacteria and fungi. Nonlimiting examples of antimicrobial and antifungal actives include b-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, triclosan; triclocarban; and mixtures thereof and the like.

Anti-wrinkle, anti-skin atrophy and skin repair actives can be effective in replenishing or rejuvenating the epidermal layer. These actives generally provide these desirable skin care benefits by promoting or maintaining the natural process of desquamation. Nonlimiting examples of antiwrinkle and anti-skin atrophy actives include vitamins, minerals, and skin nutrients such as milk, vitamins A, E, and K; vitamin alkyl esters, including vitamin C alkyl esters; magnesium, calcium, copper, zinc and other metallic components; retinoic acid and its derivatives (e.g., cis and trans); retinal; retinol; retinyl esters such as retinyl acetate, retinyl palmitate, and retinyl propionate; vitamin B 3 compounds (such as niacinamide and nicotinic acid), alpha hydroxy acids, beta hydroxy acids, e.g. salicylic acid and derivatives thereof (such as 5-octanoyl salicylic acid, heptyloxy 4 salicylic acid, and 4-methoxy salicylic acid); mixtures thereof and the like.

Skin barrier repair actives are those skin care actives which can help repair and replenish the natural moisture barrier function of the epidermis. Nonlimiting examples of skin barrier repair actives include lipids such as cholesterol, ceramides, sucrose esters and pseudo-ceramides as described in European Patent Specification No. 556,957; ascorbic acid; biotin; biotin esters; phospholipids, mixtures thereof, and the like.

Non-steroidal cosmetic soothing actives can be effective in preventing or treating inflammation of the skin. The soothing active enhances the skin appearance benefits of the present invention, e.g., such agents contribute to a more uniform and acceptable skin tone or color. Nonlimiting examples of cosmetic soothing agents include the following categories: propionic acid derivatives; acetic acid derivatives; fenamic acid derivatives; mixtures thereof and the like. Many of these cosmetic soothing actives are described in U.S. Pat. No. 4,985,459 to Sunshine et al., issued Jan. 15, 1991, incorporated by reference herein in its entirety.

Artificial tanning actives can help in simulating a natural suntan by increasing melanin in the skin or by producing the appearance of increased melanin in the skin. Nonlimiting examples of artificial tanning agents and accelerators include dihydroxyacetone; tyrosine; tyrosine esters such as ethyl tyrosinate and glucose tyrosinate; mixtures thereof, and the like.

Skin lightening actives can actually decrease the amount of melanin in the skin or provide such an effect by other mechanisms. Nonlimiting examples of skin lightening actives useful herein include aloe extract, alpha-glyceryl-L-ascorbic acid, aminotyroxine, ammonium lactate, glycolic acid, hydroquinone, 4 hydroxyanisole, mixtures thereof, and the like.

Also useful herein are sunscreen actives. A wide variety of sunscreen agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology, all of which are incorporated herein by reference in their entirety. Nonlimiting examples of sunscreens which are useful in the compositions of the present invention are those selected from the group consisting of octyl methoxyl cinnamate (Parsol MCX) and butyl methoxy benzoylmethane (Parsol 1789), 2-ethylhexyl p-methoxycinnamate, 2-ethylhexyl N,N-dimethyl-p-aminobenzoate, p-aminobenzoic acid, 2-phenylbenzimidazole-5-sulfonic acid, oxybenzone, mixtures thereof, and the like.

Sebum stimulators can increase the production of sebum by the sebaceous glands. Nonlimiting examples of sebum stimulating actives include bryonolic acid, dehydroetiandrosterone (DHEA), orizanol, mixtures thereof, and the like.

Sebum inhibitors can decrease the production of sebum by the sebaceous glands. Nonlimiting examples of useful sebum inhibiting actives include aluminum hydroxy chloride, corticosteroids, dehydroacetic acid and its salts, dichlorophenyl imidazoldioxolan (available from Elubiol), mixtures thereof, and the like.

Also useful as actives in the present invention are protease inhibitors. Protease inhibitors can be divided into two general classes: the proteinases and the peptidases. Proteinases act on specific interior peptide bonds of proteins and peptidases act on peptide bonds adjacent to a free amino or carboxyl group on the end of a protein and thus cleave the protein from the outside. The protease inhibitors suitable for use in the present invention include, but are not limited to, proteinases such as serine proteases, metalloproteases, cysteine proteases, and aspartyl protease, and peptidases, such as carboxypepidases, dipeptidases and aminopepidases, mixtures thereof and the like.

Other useful as active ingredients in the present invention are skin tightening agents. Nonlimiting examples of skin tightening agents which are useful in the compositions of the present invention include monomers which can bind a polymer to the skin such as terpolymers of vinylpyrrolidone, (meth) acrylic acid and a hydrophobic monomer comprised of long chain alkyl (meth) acrylates, mixtures thereof, and the like.

Active ingredients in the present invention may also include anti-itch ingredients Suitable examples of anti-itch ingredients which are useful in the compositions of the present invention include hydrocortisone, methdilizine and trimeprazineare, mixtures thereof and the like.

Nonlimiting examples of hair growth inhibitors which are useful in the compositions of the present invention include 17 beta estradiol, anti angiogenic steroids, curcuma extract, cycloxygenase inhibitors, evening primrose oil, linoleic acid and the like. Suitable 5-alpha reductase inhibitors such as ethynylestradiol and, genistine mixtures thereof, and the like.

Nonlimiting examples of desquamating enzyme enhancers which are useful in the compositions of the present invention include alanine, aspartic acid, N methyl serine, serine, trimethyl glycine, mixtures thereof, and the like. A nonlimiting example of an anti-glycation agent which is useful in the compositions of the present invention would be Amadorine (available from Barnet Products Distributor), and the like.

The invention will now be described in greater detail by way of the following non-limiting examples. The examples are for illustrative purposes only and not intended to limit the invention in any way. Physical test methods are described below:

Except in the operating and comparative examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

All percentages in the specification and examples are intended to be by weight unless stated otherwise.

### EXAMPLE 1

A series of inventive examples were made according to Table 1 using the procedure below in order to evaluate the effect of N—(C₆-C₂₀) acyl Sarcosinate surfactants and petrolatum amounts and ratio on foaming and stability. The foaming and stability properties were determined using the procedures provided below and the results are summarized in Table 1. It was found that the inventive formulations provided overall superior foaming and stability compared to the comparative formulations listed in Example 4.

TABLE 1

| | % by wt. | | | | | |
|---|---|---|---|---|---|---|
| Type product (1) | BW | BW | BW | BW | Face | Face |
| Identification | 098 | 099 | 224 | 096 | FII | J53 |
| Surfactants (total) | 14.00 | 16.00 | 10.50 | 14.00 | 11.00 | 14.00 |
| Amonium Lauryl Sulfate | 2.9 | 2.9 | 2.34 | 2.63 | | |
| Amonium Laureth Sulfate | 2.33 | 2.33 | 1.83 | 2.06 | | |
| PEG Cocamide MEA | .255 | .255 | .20 | .45 | | |
| Cocomidopropyl Betaine | 2.00 | 2.00 | 2.00 | 2.00 | | |
| Cocamide MEA | .51 | .51 | .40 | .23 | | |
| Sodium Lauroyl Sarcosinate | 3.00 | 3.00 | 0.50 | 3.00 | 3.00 | 6.00 |
| Steol 170 (Sodium Lauryl | | | | | | |
| Sulfonate) | | | | | | |
| Disodium Lauryl Sulfosuccinate | | | | | 8.00 | 8.00 |
| Fatty Acids (total) | 3.3 | 2.00 | 2.30 | 1.30 | 4.80 | 4.80 |
| Lauric Acid | 3.3 | 2.00 | 2.30 | 1.30 | 4.00 | 4.00 |
| Stearic Acid | | | | | 0.80 | 0.80 |
| Polymers/Structurants (total) | 4.85 | 2.35 | 2.69 | 3.55 | 4.18 | 4.18 |
| Lauryl alcohol | 0.50 | 0.50 | 0.50 | 0.50 | | |
| Starch B990 (Hydoxypropyl | 4.00 | 1.50 | 3 | 3.00 | 3.00 | 3.00 |
| Starch Phosphate) | | | | | | |
| Guar Hydroxypropyltrimonium | 0.35 | 0.35 | 0.15 | 0.35 | | |
| Chloride | | | | | | |
| Styleze W-20 (Pq-55) | | | | 0.05 | 1.00 | 1.00 |
| Polyquaternium-39 | | | | | 0.16 | 0.16 |
| Polyquaternium-7 | | | | | 0.02 | 0.02 |
| Peg 14M | 0.15 | 0.15 | 0.05 | 0.15 | | |
| Emollients (total) | 24.00 | 44.00 | 8.50 | 43.00 | 35.00 | 35.00 |
| Petrolatum | 20.00 | 40.00 | 4.00 | 40.00 | 5.00 | 5.00 |
| Glycerin | | | 3.00 | | 30.00 | 30.00 |
| Soybean Oil | 2.00 | 1.00 | 1.00 | | | |
| Indopol H300/1500 | 2.00 | 3.00 | 0.50 | 3.00 | | |
| (Polybutene) | | | | | | |
| Water, dyes, fragrance etc. to | 100.00 | 100.00 | 100.00 | 100.00 | 100.00 | 100.00 |
| 100% | | | | | | |
| Viscosity, KPaS (2) | 79.2** | 83.2** | 95.0** | 90.0** | 110.0* | 90.0* |
| pH | 6.3 | 6.6 | 6.2 | 6.3 | 5.5 | 5.7 |
| Stability (3) | acceptable | acceptable | acceptable | acceptable | acceptable | acceptable |
| Foam volume (4) | 28 ml | 30 ml | 27 ml | 33 ml | 35 ml | 40 ml |
| Mildness & Moisturization | 4.5 | 6.5 | 3.0 | 6.0 | 6.5 | 6.0 |
| Rating (5) | | | | | | |

Notes:
(1) BW denotes a body wash and Face denotes a facial cleanser.
(2) Viscosity method * S-7 10 rpm DVT/** .5 rpm T-A RVT
(3) Testing cycle: 12 wks 45 C. constant/cycle 10 × −10 C./25 C./day
(4) See procedure below.
(5) See procedure below.

### EXAMPLE 2

A series of inventive examples were made according to Table 2 using the procedure below in order to evaluate the effect of acyl Sarcosinate fatty acid alkyl number on foaming and stability. The foaming and stability properties were determined using the procedures provided below and the results are summarized in Table 2. It was found that Sodium Lauroyl Sarcosinate, Sodium Myristoyl Sarcosinate and Sodium Cocoyl Sarcosinate all provided superior foaming and stability.

TABLE 2

| | % by wt. | | |
|---|---|---|---|
| Type product | BW | BW | BW |
| Identification | 341 | 355 | 357 |
| Surfactants (total) | 14.00 | 14.00 | 14.00 |
| Ammonium Lauryl Sulfate | 2.63 | 2.63 | 2.63 |
| Ammonium Laureth Sulfate | 2.06 | 2.06 | 2.06 |
| PEG Cocamide MEA | .45 | .45 | .45 |
| Cocamidopropyl Betaine | 2.00 | 2.00 | 2.00 |
| Cocamide MEA | .23 | .23 | .23 |
| Sodium Lauroyl Sarcosinate | 3.00 | | |
| Sodium Myristoyl Sarcosinate | | 3.00 | |
| Sodium Cocoyl Sarcosinate | | | 3.00 |
| Steol 170 (Sodium Lauryl Sulfonate) | | | |
| Disodium Lauryl Sulfosuccinate | | | |
| Fatty Acids (total) | 2.50 | 1.50 | 1.50 |
| Lauric Acid | 2.50 | 1.50 | 1.50 |
| Stearic Acid | | | |
| Polymers/Structurants (total) | 3.50 | 2.50 | 2.50 |
| Lauryl alcohol | 0.50 | 0.50 | 0.50 |
| Starch B990 (Hydoxypropyl Starch | 3.00 | 2.00 | 2.00 |
| Phosphate) | | | |
| Guar Hydroxypropyltrimonium | 0.35 | 0.35 | 0.35 |
| Chloride | | | |
| Structure XL (Hydroxypropyl Starch | | | |
| Phosphate) | | | |
| Styleze W-20 (Pq-55) | | | |
| Polyquaternium-39 | | | |
| Polyquaternium-7 | | | |
| Peg 14 M | 0.15 | 0.15 | 0.15 |
| Emollients (total) | 22.00 | 22.00 | 22.00 |
| Petrolatum | 20.00 | 20.00 | 20.00 |
| Glycerin | | | |
| Soybean Oil | | | |
| Indopol H300/1500 (Polybutene) | 2.00 | 2.00 | 2.00 |
| Water, dyes, fragrance etc. to 100 | 100.00 | 100.00 | 100.00 |
| Viscosity KPaS | 79.2** | 80.0** | 85.0** |
| pH | 6.3 | 6.4 | 6.5 |
| Stability | acceptable | acceptable | acceptable |
| Foam volume | 28 ml | 25 ml | 23 ml |
| Mildness & Moisturization Rating | 4.5 | 4.5 | 4.5 |

### EXAMPLE 3

A series of inventive examples were made according to Table 3 using the procedure below in order to evaluate the effect of petrolatum samples having different melting point ranges on foaming and stability. The foaming and stability properties were determined using the procedures provided below and the results are summarized in Table 3. It was found that the petrolatum materials tested all provided superior foaming and stability.

TABLE 3

| Type product | BW | BW | Face | Face |
|---|---|---|---|---|
| Identification | 096 | 096a | Fire II | Fire IIa |
| Surtactants (total) | 14.00 | 14.00 | 11.00 | 11.00 |
| Ammonium Lauryl Sulfate | 2.63 | 2.63 | | |
| Ammonium Laureth Sulfate | 2.06 | 2.06 | | |
| PEG Cocamide MEA | .45 | .45 | | |
| Cocamidopropyl Betaine | 2.00 | 2.00 | | |
| Cocamide MEA | .23 | .23 | | |
| Sodium Lauroyl Sarcosinate | 3.00 | 3.00 | 3.00 | 3.00 |
| Steol 170 (Sodium Lauryl Sulfonate) | | | | |
| Disodium Lauryl Sulfosuccinate | | | 8.00 | 8.00 |
| Fatty Acids/Structurants | 1.30 | 1.30 | 4.80 | 4.80 |
| Lauric Acid | 1.30 | 1.30 | 4.00 | 4.00 |
| Stearic Acid | | | 0.80 | 0.80 |
| Polymers/Structurants (total) | 3.55 | 3.55 | 4.18 | 4.18 |
| Lauryl alcohol | 0.50 | 0.50 | | |
| Starch B990 (Hydroxypropyl Starch | 3.00 | 3.00 | 3.00 | 3.00 |
| Phosphate) | | | | |
| Guar Hydroxypropyltrimonium Chloride | 0.35 | 0.35 | | |
| Structure XL (Hydroxypropyl Starch | | | | |
| Phosphate) | | | | |
| Styleze W-20 (Pq-55) | 0.05 | 0.05 | 1.00 | 1.00 |
| Polyquaternium-39 | | | 0.16 | 0.16 |
| Polyquaternium-7 | | | 0.02 | 0.02 |
| Peg 14 M | 0.15 | 0.15 | | |
| Emollients (total) | 43.00 | 43.00 | 35.00 | 35.00 |
| Petrolatum “snow white” Penreco | 40.00 | | 5.00 | |
| Petrolatum G-1937 (2.5 Hard) Witco | | 40.00 | | 5.00 |
| Glycerin | | | 30.00 | 30.00 |
| Soybean Oil | | | | |
| Indopol H300/1500 (Polybutene) | 3.00 | 3.00 | | |
| Water, dyes, fragrance etc. to 100 | 100.00 | 100.00 | 100.00 | 100.00 |
| Viscosity (KPaS) | 90.0** | 60.0** | 110.0* | 75.0* |
| pH | 6.3 | 6.3 | 5.5 | 5.5 |
| Stability | acceptable | acceptable | acceptable | acceptable |
| Foam volume | 33 ml | 20 ml | 35 ml | 26 ml |
| Mildness & Moisturization Ranking | 6.5 | 6.5 | 6.5 | 6.5 |

| Identification | Trade Name | Source | Melting Point Range |
|---|---|---|---|
| Petrolatum G-1937 Witco | 2.5 Hard | Witco | 38 C.–60 C. |
| Petrolatum | Snow White | Penreco | 51.7 C.–57.2 C. |

### EXAMPLE 4

A series of comparative examples were made according to Table 4 using the procedure below in order to evaluate the effect of the absence of N—(C₆-C₂₀) acyl Sarcosinate surfactants or where the sarcosinate and petrolatum ratios were outside the inventive range or where the C10-C18 fatty acid concentrations were outside the inventive range. The foaming and stability properties were determined using the procedures provided below and the results are summarized in Table 4. It was found that the comparative samples tested all provided poor foaming and/or stability compared to the inventive cases shown above.

TABLE 4

| Product Type | BW | Face | Face | Face | Face | BW |
|---|---|---|---|---|---|---|
| Identification | 06-1 | 06-6 | 416 | 417 | 418 | 06-5 |
| Surfactants (total) | 11.33 | 8.33 | 14.00 | 11.00 | 11.00 | 13.00 |
| Ammonium Lauryl Sulfate | 2.63 | | 1.75 | | | 2.63 |
| Ammonium Laureth Sulfate | 2.06 | | 1.37 | | | 2.06 |
| PEG Cocamide MEA | .45 | | .30 | | | .45 |
| Cocamidopropyl Betaine | 2.00 | | | 3.00 | | 2.00 |
| Cocamide MEA | .23 | | .15 | | | 2.23 |
| Sodium Lauroyl Sarcosinate | 0.33 | 0.33 | 0.00 | 0.00 | 0.00 | 0.00 |
| Steol 170 (Sodium Lauryl Sulfonate) | | | | | 3.00 | |
| Disodium Lauryl Sulfosuccinate | | 8.00 | 8.00 | 8.00 | 8.00 | |
| Fatty Acids/Alcohols “Structurants” | 3.00 | 4.80 | 4.80 | 4.80 | 4.80 | 4.80 |
| (total) | | | | | | |
| Lauric Acid | 2.50 | 4.00 | 4.00 | 4.00 | 4.00 | 4.00 |
| Stearic Acid | | 0.80 | 0.80 | 0.80 | 0.80 | 0.80 |
| Lauryl alcohol | 0.50 | | | | | |
| Polymers/Structurants (total) | 3.50 | 4.18 | 39.18 | 44.18 | 39.18 | 0.75 |
| Starch B990 (Hydroxypropyl Starch | 3.00 | 3.00 | 3.00 | 3.00 | 3.00 | 0.25 |
| Phosphate | | | | | | |
| Guar Hydroxypropyltrimonium Cl | 0.35 | | | | | 0.35 |
| Polyquaternium-55 | | 1.00 | 1.00 | 1.00 | 1.00 | |
| Polyquaternium-39 | | 0.16 | 0.16 | 0.16 | 0.16 | |
| Polyquaternium-7 | | 0.02 | 0.02 | 0.02 | 0.02 | |
| Peg 14M | 0.15 | | | | | 0.15 |
| Emollients (total) | 42.00 | 70.00 | 35.00 | 40.00 | 35.00 | 23.00 |
| Petrolatum | 40.00 | 50.00 | 5.00 | 10.00 | 5.00 | 20.00 |
| Glycerin | | 20.00 | 30.00 | 30.00 | 30.00 | |
| Soybean Oil | | | | | | |
| Indopol H300/1500 (Polybutene) | 2.00 | | | | | 3.00 |
| Water, dyes, etc. to | 100.00 | 100.00 | 100.00 | 100.00 | 100.00 | 100.00 |
| Viscosity (KPaS) | 79.2** | 95.0** | 70.0* | 80.0* | 67.0* | 35.0** |
| pH | 6.3 | 6.2 | 6.1 | 6.4 | 6.1 | 6.6 |
| Stability | acceptable | acceptable | acceptable | acceptable | acceptable | acceptable |
| Foam volume | 15 ml | 17 ml | 10 ml | 5 ml | 10 ml | 19 ml |
| Mildness & Moisurization Ranking | 7.0 | 8.0 | 6.5 | 6.0 | 6.5 | 4.5 |

### EXAMPLE 5

A series of comparative examples were made according to Table 5 using the procedure below in order to evaluate the effect where the N—(C₆-C₂₀) acyl Sarcosinate surfactants and petrolatum ratios were outside the inventive range. The foaming and stability properties were determined using the procedures provided below and the results are summarized in Table 5. It was found that the comparative samples tested all provided poor foaming and/or stability compared to the inventive cases shown above.

TABLE 5

| Product Type | BW | Face | Face |
|---|---|---|---|
| Identification | 06-1 | 06-5 | 06-6 |
| Surfactants (total) | 11.33 | 16.00 | 8.33 |
| Amonium Lauryl Sulfate | 2.63 | | |
| Amonium Laureth Sulfate | 2.06 | | |
| PEG Cocamide MEA | .45 | | |
| Cocamidopropyl Betaine | 2.00 | | |
| Cocamide MEA | .23 | | |
| Sodium Lauroyl Sarcosinate | 0.33 | 8.00 | 0.33 |
| Steol 170 (Sodium Lauryl Sulfonate) | | | |
| Disodium Lauryl Sulfosuccinate | | 8.00 | 8.00 |
| Fatty Acids/Alcohols “Structurants” | 3.00 | 4.80 | 4.80 |
| (Total) | | | |
| Lauric Acid | 2.50 | 4.00 | 4.00 |
| Stearic Acid | | 0.80 | 0.80 |
| Lauryl alcohol | 0.50 | | |
| Polymers/Structurants (total) | 3.50 | 2.18 | 4.18 |
| Starch B990 (Hydroxypropyl Starch | 3.00 | 1.00 | 3.00 |
| Phosphate) | | | |
| Guar Hydroxypropyltrimonium Cl | 0.35 | | |
| Polyquaternium-55 | | 1.00 | 1.00 |
| Polyquaternium-39 | | 0.16 | 0.16 |
| Polyquaternium-7 | | 0.02 | 0.02 |
| Peg 14 M | 0.15 | | |
| Emollients (total) | 42.00 | 31.00 | 60.00 |
| Petrolatum | 40.00 | 1.00 | 40.00 |
| Glycerin | | 30.00 | 20.00 |
| Soybean Oil | | | |
| Indopol H300/1500 (Polybutene) | 2.00 | | |
| Water, dyes, etc. to | 100.00 | 100.00 | 100.00 |
| Viscosity (KPaS) | 79.2** | 54.4** | 95.0** |
| pH | 6.3 | 6.4 | 6.2 |
| Stability | acceptable | acceptable | acceptable |
| Foam volume | 15 ml | 27 ml | 17 ml |
| Mildness & Moisturization Ranking | 7.0 | 4.0 | 7.0 |

### EXAMPLE 6

A series of comparative examples were made according to Table 6 using the procedure below in order to evaluate the effect where the C₁₀-C₁₈) fatty acids were outside the inventive concentration range. The foaming and stability properties were determined using the procedures provided below and the results are summarized in Table 6. It was found that the comparative samples tested all provided poor foaming and/or stability compared to the inventive cases shown above.

TABLE 6

| Product Type | BW | Face |
|---|---|---|
| Identification | BC712-1 | BC712-2 |
| Surfactants (total) | 16.00 | 13.00 |
| Amonium Lauryl Sulfate | 2.63 | |
| Amonium Laureth Sulfate | 2.06 | |
| PEG Cocamide MEA | .45 | |
| Cocamidopropyl Betaine | 2.00 | |
| Cocamide MEA | 2.23 | |
| Sodium Lauroyl Sarcosinate | 3.00 | 3.00 |
| Steol 170 (Sodium Lauryl Sulfonate) | | |
| Sodium cocoyl glycinate | | 2.00 |
| Disodium Lauryl Sulfosuccinate | | 8.00 |
| Fatty Acids/Alcohols “Structurants” | 0.30 | 0.40 |
| (total) | | |
| Lauric Acid | 0.30 | 0.20 |
| Stearic Acid | | 0.20 |
| Lauryl alcohol | | |
| Polymers/Structurants (total) | 0.75 | 0.85 |
| Starch B990 (Hydroxypropyl Starch | 0.25 | |
| Phosphate) | | |
| Guar Hydroxypropyltrimonium Cl | 0.35 | 0.35 |
| Polyquaternium-55 | | 0.50 |
| Polyquaternium-39 | | |
| Polyquaternium-7 | | |
| Peg 14 M | 0.15 | |
| Emollients (total) | 23.00 | 45.00 |
| Petrolatum | 20.00 | 5.00 |
| Glycerin | | 40.00 |
| Soybean Oil | | |
| Indopol H300/1500 (Polybutene) | 3.00 | |
| Water, dyes, etc. to | 100.00 | 100.00 |
| Viscosity (KPaS) | 35.0** | 20.0** |
| pH | 6.6 | 6.8 |
| Stability | split 1 wk 45 C | split 2 wk 45 C |
| | (fail) | (fail) |
| Foam volume | 30 ml | 35 ml |
| Mildness & Moisturization Rating | 6.5 | 6.5 |

### EXAMPLE 7

A series of comparative examples were made according to Table 7 using the procedure below in order to evaluate the effect where no sarcosinate surfactants were used. The foaming and stability properties were determined using the procedures provided below and the results are summarized in Table 7. It was found that the comparative samples tested all provided poor foaming and/or stability compared to the inventive cases shown above

TABLE 7

| | Face | Face | Face | BW | BW | BW |
|---|---|---|---|---|---|---|
| Identification | 416 | 417 | 418 | 06-5 | 06-5 | 06-5 |
| Surfactants (total) | 14.00 | 11.00 | 11.00 | 13.00 | 13.00 | 13.00 |
| Ammonium Lauryl Sulfate | 1.75 | | | 2.63 | 2.63 | 2.63 |
| Ammonium Laureth Sulfate | 1.37 | | | 2.06 | 2.06 | 2.06 |
| PEG Cocamide MEA | .25 | | | .45 | .45 | .45 |
| Cocamidopropyl Betaine | | 3.00 | | 2.00 | 2.00 | 2.00 |
| Cocamide MEA | .30 | | | 2.23 | 2.23 | 2.23 |
| Sodium Lauroyl Sarcosinate | 0.00 | 0.00 | 0.00 | 0.00 | 0.00 | 0.00 |
| Steol 170 (Sodium Lauryl Sulfonate) | | | 3.00 | | | |
| Disodium Lauryl Sulfosuccinate | 8.00 | 8.00 | 8.00 | | | |
| Fatty Acids/Alcohols “Structurants” (total) | 4.80 | 4.80 | 4.80 | 4.80 | 4.80 | 4.80 |
| Lauric Acid | 4.00 | 4.00 | 4.00 | 4.00 | 4.00 | 4.00 |
| Stearic Acid | 0.80 | 0.80 | 0.80 | 0.80 | 0.80 | 0.80 |
| Polymers/Structurants (total) | 39.18 | 44.18 | 39.18 | 1.50 | 0.75 | 0.75 |
| Starch B990 (Hydroxypropyl Starch | 3.00 | 3.00 | 3.00 | 1.00 | 0.25 | 0.25 |
| Phosphate) | | | | | | |
| Guar Hydroxypropyltrimonium Cl | | | | 0.35 | 0.35 | 0.35 |
| Polyquaternium-55 | 1.00 | 1.00 | 1.00 | | | |
| Polyquaternium-39 | 0.16 | 0.16 | 0.16 | | | |
| Polyquaternium-7 | 0.02 | 0.02 | 0.02 | | | |
| Peg 14M | | | | 0.15 | 0.15 | 0.15 |
| Emollients (total) | 35.00 | 40.00 | 35.00 | 23.00 | 23.00 | 23.00 |
| Petrolatum | 5.00 | 10.00 | 5.00 | 20.00 | 20.00 | 20.00 |
| Glycerin | 30.00 | 30.00 | 30.00 | | | |
| Soybean Oil | | | | | | |
| Indopol H300/1500 (Polybutene) | | | | 3.00 | 3.00 | 3.00 |
| Water, dyes, etc. to | 100.00 | 100.00 | 100.00 | 100.00 | 100.00 | 100.00 |
| Viscosity (KPaS) | 70.0* | 80.0* | 67.0* | 35.0** | 35.0** | 35.0** |
| pH | 6.1 | 6.4 | 6.1 | 6.6 | 6.6 | 6.6 |
| Stability | acceptable | acceptable | acceptable | acceptable | acceptable | acceptable |
| Foam volume | 10 ml | 5 ml | 10 ml | 19 ml | 19 ml | 19 ml |
| Mildness & Moisturization Ranking | 6.0 | 6.0 | 5.5 | 4.5 | 4.5 | 4.5 |

### EXAMPLE 8

A series of inventive and comparative examples were made according to Table 8 using the procedure below in order to evaluate the effect of the presence or absence of inventive sarcosinate surfactants. The foaming and stability properties were determined using the procedures provided below and the results are summarized in Table 8. It was found that the comparative samples tested all provided poor foaming and/or stability compared to the inventive cases shown adjacent to the comparative cases.

TABLE 9

| Identification | Trade Name | Source |
|---|---|---|
| INCI NAME | | |
| Cocamidopropyl Betaine | Tegobetane F | Dequssa |
| Cocamide MEA | Ninol Comf | Stepan |
| Sodium Lauroyl Sarcosinate | Hamposyl L-30 | Chattem Chemicals |
| Sodium Cocoyl Sarcosinate | Hamposyl C-30 | Chattem Chemicals |
| Sodium Myristoyl Sarcosinate | Hamposyl M-30 | Chattem Chemicals |
| Sodium Lauryl Sulfonate | Steol 170 | Stepan |
| Sodium Laureth Sulfate | Steol CS370 | Stepan |
| Na cocoyl glycinate | Amilite GCS-11 | Ajinomoto |
| Disodium Lauryl Sulfosuccinate | Mackanate LO-100 | Mclntyre Group |
| Fatty Acids/Structurants | | |
| Palmitic Acid | Prific 2960 | Uniqema |
| Lauric Acid | Prifrac 2922 | Uniqema |
| Stearic Acid | Pristerene 4911 | Uniqema |
| Polymers/Structurants | | |
| Lauryl Alcohol | Lorol C12 14 A | Cognis |
| Hydroxypropyl Starch Phosphate | Pure Gel B990 | Grain Processing |
| Guar Hydroxypropyltrimonium Chloride | Jaguar C13S | Rhodia |
| Hydroxypropyl Starch Phosphate | Structure XL | National Starch |
| Polyquaternium-55 | Styleze W-20 | ISP |
| Polyquaternium-39 | Merquat 3330 | Nalco |
| Polyquaternium-7 | Merquat 550 | Nalco |
| Peg 14 M | Polyox WSR N-300 | Amerchol |
| Peg-30 Dipolyhydroxy Stearate | Arlacel P-135 | Uniqema |
| Emollients | | |
| Petrolatum G-1937 Witco | 2.5 Hard | Witco |
| Petrolatum | Snow White | Penreco |
| Glycerin | Pricerine 9088 | Uniqema |
| Soybean Oil | Refined Soybean Oil | Welch Home Clark |
| Indopol H300/1500 | Polybutene | ISP |

TABLE 8

| Product Type | BW | BW | BW | BW | BW | BW | Face | Face |
|---|---|---|---|---|---|---|---|---|
| Identification | BD06-047 | BD06-048 | BD06-024 | BD06-025 | BD06-605 | BD06-606 | BC347 | 8287 |
| Surfactants (total) | 29.30 | 17.00 | 18.80 | 18.80 | 20.50 | 20.50 | 11.00 | 11.00 |
| Cocamidopropyl Betaine | 4.00 | 4.00 | 4.00 | 4.00 | 5.70 | 5.70 | | |
| Cocamide MEA | 3.00 | 3.00 | 2.50 | 2.50 | 2.50 | 2.50 | | |
| Sodium Lauroyl Sarcosinate | | 10.00 | | 6.00 | | 6.00 | | 3.00 |
| Steol 170 (Sodium Lauryl | 10.00 | | | | | | | |
| Sulfonate) | | | | | | | | |
| Steol CS370 | 12.30 | | 12.30 | 6.30 | 12.30 | 6.30 | | |
| Na cocoyl glycinate | | | | | | | 3.00 | |
| Disodium Lauryl Sulfosuccinate | | | | | | | 8.00 | 8.00 |
| Fatty Acids/Structurants (total) | 3.00 | 3.00 | 3.30 | 3.30 | 2.90 | 2.90 | 4.80 | 4.80 |
| Palmitic Acid | | | | | | | | |
| Lauric Acid | 3.00 | 3.00 | 3.30 | 3.30 | 2.90 | 2.90 | 4.00 | 4.00 |
| Stearic Acid | | | | | | | 0.80 | 0.80 |
| Polymers/Structurants (total) | 0.60 | 0.60 | 0.70 | 0.70 | 0.95 | 0.95 | 4.18 | 4.18 |
| Starch B990 (Hydroxypropyl | | | | | | | | |
| Starch Phosphate) | | | | | | | | |
| Guar Hydroxypropyltrimonium | 0.35 | 0.35 | 0.70 | 0.70 | 0.70 | 0.70 | | |
| Chloride | | | | | | | | |
| Structure XL (Hydroxypropyl | | | | | | | 3.00 | 3.00 |
| Starch Phosphate) | | | | | | | | |
| Styleze W-20 (Pq-55) | | | | | | | 1.00 | 1.00 |
| Polyquaternium-39 | | | | | | | 0.16 | 0.16 |
| Polyquaternium-7 | | | | | | | 0.02 | 0.02 |
| Peg 14M | | | | | | | | |
| Arlacel P-135 (Peg-30 | 0.25 | 0.25 | | | 0.25 | 0.25 | | |
| Dipolyhydroxy Stearate | | | | | | | | |
| Emollients (total) | 20.25 | 20.25 | 23.95 | 23.95 | 17.70 | 17.70 | 35.00 | 35.00 |
| Petrolatum | 5.00 | 5.00 | 3.70 | 3.70 | 3.70 | 3.70 | 5.00 | 5.00 |
| Glycerin | 4.00 | 4.00 | 5.70 | 5.70 | 7.00 | 7.00 | 30.00 | 30.00 |
| Soybean Oil | 11.00 | 11.00 | 14.30 | 14.30 | 7.00 | 7.00 | | |
| Indopol H300/1500 | 0.25 | 0.25 | 0.25 | 0.25 | | | | |
| (Polybutene) | | | | | | | | |
| Water, dyes, fragrance etc. to | 100.00 | 100.00 | 100.00 | 100.00 | 100.00 | 100.00 | 100.00 | 100.00 |
| 100 | | | | | | | | |
| Viscosity (KPaS) | 145.0** | 100.0** | 62.8** | 50.0** | 96.0** | 83.0** | 180.0* | 95.0* |
| pH | 5.45 | 5.77 | 5.42 | 5.61 | 5.61 | 6 | 5.5 | 5.7 |
| Stability | acceptable | acceptable | acceptable | acceptable | acceptable | acceptable | acceptable | acceptable |
| Foam volume | 21 ml | 37 ml | 25 ml | 34 ml | 20 ml | 37 ml | 23 | 38 |
| Mildness & Moisturization | 3.5 | 4.0 | 3.5 | 4.0 | 4.5 | 4.0 | 5.0 | 4.5 |
| Ranking | | | | | | | | |

Examples were prepared with the following materials listed in table 9 below:

Procedure for Sample Preparation of BW Type Products
1 Add approx. 65% portion of water and Lauric Acid to main vessel. Begin heating to 70C
2 Add Soybean Oil
3 Add Guar
4 Add about 12.5% of total Petrolatum (PJ) and maintain heat at 70C until dissolved.
5 Add Betaine; maintain heat at 70C
6 In a side tank add starch and add 4 times its weight in water and mix gently. Then add to main vessel.
7 Add Almeo (1) blend heat to 70C
8 Add approx. 20% of water
9 Add sarcosinate
10 Add Lauryl Alcohol
11 Cool to 45 C and add preservatives and fragrance mix 15 min
12 Add Indopol/remaining petrolatum at about 42 C mix and cool to 40 C
(1) Almeo is the trade name for a blend surfactants purchased from Stepan Company It contains=29.25% Ammonium Lauryl Sulfate; 22.85% Ammonium Laureth Sulfate; 5.0% Cocamide MEA; 2.5% PEG 5 Cocamide; and 39.85% water

Procedure for Sample Preparation of Face Type Products.
1 Add approx. 80% water, EDTA & Glycerin to main tank and begin heating.
2When at 35 C slowly add starch to main mixing tank
3 Mix until homogeneous
4 Use a side tank and mix Stylize® with 8× it's weight in water at 22-25 C
5 Add Styleze® water to main tank when homogenous let mix for 10 min
6 Add Merquats® to main tank and mix until homogeneous
7 Add Surfactants to main tank heat until dissolved around 55 C.
8 Weigh fatty acids and PJ together in side tank melt with sufficient heat and mix
9 When fatty acids and main vessel are both at 60 C add fatty acids and PJ to main tank and mix for 15 min.
10 Let cool to 35 C add fragrance and preservative.
11 Let cool.

### Methods:

a. Foam Determination Method:
1. Dilute product in a 1/10 solution (i.e. 10 g product to 100 ml deionized water at 22 to 25 C).
2. Wet hand with water at 22 to 25 C. Place 2.5 ml of the diluted product on the wet hands and rub 1 0 times in a circular motion. Collect foam lather in a tared dish. Repeat with second 2.5 mls of diluted product. Combine lather in dish and weigh. Calculate weight of foam collected.
3. Collect measured volume (e.g. 1.5 mis) of foam lather in a tared Petri dish and weigh. Calculate density as follows:

Density of foam=weight of foam/volume of foam
4. Calculate volume of foam collected in step 2 as follows:

Foam lather volume=weight of total foam/density of foam

### B. Standardized Stability Tests:

Samples are stored at the following conditions and evaluated (see note 1 below) at the following evaluation points using the cycle and constant temperature tests outlined below.

### Stability Test Conditions:

| tests | Time | Evaluation Points |
|---|---|---|
| Room Temp (RT) | 10 days | After 10 cycles |
| approx. 22 C. to | | |
| 40 C. cycle over 1 | | |
| day | | |
| −10 C. to 25 C. cycle | 10 days | After 10 cycles |
| over 1 day | | |
| 5 C. to 25 C. cycle | 60 days | After 60 cycles |
| over 1 day | | |
| Approx. 22 C | 12 weeks | Initial, overnight |
| constant temp | | 4, 8, 12 week |
| | | intervals. |
| 37 C. constant | 12 weeks | 2, 4, 8, 12 week |
| temp | | intervals. |
| 45 C. constant | 12 weeks | 2, 4, 8, 12 week |
| temp | | intervals. |

Note 1:
Viscosity, pH and visual appearance are evaluated for the test sample as follows.
Viscosity: Measured by the method indicated for each example (see examples).
pH: Measured by Accumet Research AR 15 pH Meter.
Visual evaluation: color, odor, and appearance by inspection.

A sample is considered stable if its viscosity (i.e. greater than 20% relative) and visual appearance do not change significantly from the initial measurements at all stability test conditions described in (b) above.

### C. Standard Viscosity Method:

Scope:

This method covers the measurement of the viscosity of a preferred embodiment of the invention that has an ordered liquid crystalline phase.

Apparatus:
Brookfield RVT or DVT Viscometer with digital readout and Helipath Accessory; Chuck, weight and closer assembly for T-bar attachment; T-bar Spindle A for RVT and S-7 spindle for DVT.
Plastic cups diameter greater than 2.5 inches.

Procedure:
1. Verify that the viscometer and the helipath stand are level by referring to the bubble levels on the back of the instrument.
2. connect the chuck/closer/weight assembly to the Viscometer (Note the left-hand coupling threads).
3. Clean Spindle A with deionized water and pat dry with a Kimwipe sheet. Slide the spindle in the closer and tighten.
4. Set the rotational speed at 0.5 RPM for T bar spindle or 10 rpm for S-7 spindle. In case of a digital viscometer (DV) select the % mode and press autozero with the motor switch on.
5. Place the product in a plastic cup with inner diameter of greater than 2.5 inches. The height of the product in the cup should be at least 3 inches. The temperature of the product should be 25° C.
6. Lower the spindle into the product (˜¼ inches). Set the adjustable stops of the helipath stand so that the spindle does not touch the bottom of the plastic cup or come out of the sample.
7. Start the viscometer and allow the dial to make one or two revolutions before turning on the Helipath stand. Note the dial reading as the helipath stand passes the middle of its traverse.
8. Record reading in centipoise.

D. Method for Determining Low Shear Viscosity

Equipment Used, Advanced Rheometer AR 1 000
Cone=40 mm, 20 steal
Cone angle=1:59:38 (deg:min:sec:)
Gap=48 micro m
Shear Rate=10 sec-1
Interval=1 min.

### Procedure

1, Add correct cone to instrument,

2, Power up instrument:

3, Set instrument to above settings:

4, Place 5 ml of test product under cone on sample platform.

5, Start instrument and record results.

E. Mildness & Moisturization Test

Panelists are selected from persons aged 39 years and older.

Pre-treatment: Wash forearms with Ivory® soap bar twice a day (30-second washes) during the 6-day conditioning period.

Pre-Product Application: Both the subject's inner forearms are divided and marked with a skin marking pen and Scanpor®-like tape into four (3×3 cm square) test sites (2×2) placed approx. 5 cm from the arm flex area and from the wrist, for a total of eight test sites per subject according to the following diagram.

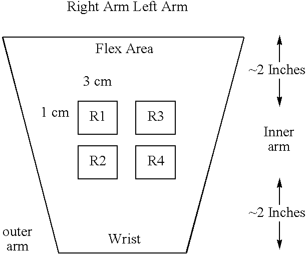

Product Application: Six of the test sites are washed twice a day (30-second washes) during the 4-day testing period with designated test products.

Control Application: One site on each arm is utilized for a control and the control is applied the same time as the test product.

Controls: Two controls are utilized.

Positive Control: Vaseline® Intensive Care® lotion (Unilever, Greenwich Conn.) was applied to one site and left on the site throughout the application period twice a day at the same time as the test products.

Negative Control: Further washing with Ivory® soap on one site throughout the application period.

Mildness and Moisturization (M&M) Grading: An expert grader was utilized to grade relative M&M one day after the last product application. The positive control site was arbitrarily ranked as a 10 and the negative control site was arbitrarily ranked as a 0.

Evaluation of results: The procedure resulted in individual test products having a designated Mildness and Moisturization ranking on a scale of 0-10 were 10 was a ultra mild and moisturizing and 0 was harsh and drying.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention

We claim:

1. A stable, liquid personal cleansing composition, comprising:
a. about 0.5 to 20% by wt. of total N—(C₆-C₂₀) acyl sarcosinate surfactant(s);
b. about 0.5 to 50% by wt. of total hydrocarbon oil or wax emollients or blends thereof;
c. greater than about 0.5% by wt. of C10 to C18 fatty acid(s);
d. about 5 to 95% by wt. of water; and
e. wherein the ratio of the sarcosinate surfactant to total hydrocarbon wax and oil emollients is in the range of about 0.04 to 2.0.

2. The composition of claim 1, wherein the cleansing composition has a liquid crystal structured phase.

3. The composition of claim 1, wherein the viscosity value is in the range of about 8 KPaS to 800 KPaS at 25 C using the Standard Viscosity Method.

4. The composition of claim 1 further comprising greater than about 0.5% by wt. of hydrophobic emollient(s) selected from glyceride oil(s), polybutenes with a number average degree of polymerization of about 3 to about 110, silicone oils and blends thereof.

5. The cleansing composition of claim 1 wherein the total hydrocarbon wax and oil emollients blend has an observed melting point in the range of about 35 to 70 C.

6. The cleansing composition of claim 1 wherein the foam volume is greater than or equal to 20 mis using the standard foam determination method.

7. The cleansing composition of claim 1 further comprises about 3 to 30% by wt. of total anionic, amphoteric and cationic surfactant(s) or blends thereof not including the N—(C₆-C₂₀) acyl sarcosinate surfactant(s).

8. The cleansing composition of claim 1 further comprising about 0.05 to 10% by wt. of cationic polymer(s).

9. The cleansing composition of claim 1 wherein the composition remains stable under at least one of the Standardized Stability tests.
